(19) United States
(12) Patent Application Publication
(10) Pub. No.: US 2022/0163521 A1
(43) Pub. Date: May 26, 2022

(54) PAPER LATERAL FLOW IMMUNOASSAY

(71) Applicant: Revelant Labs, Provo, UT (US)

(72) Inventors: Annie Pitts ARMITSTEAD, Provo, UT (US); Lara GRETHER, Provo, UT (US); Richard WATT, Provo, UT (US); Kelly PITTS, Provo, UT (US)

(21) Appl. No.: 17/597,448

(22) PCT Filed: Jul. 7, 2020
(86) PCT No.: PCT/US2020/041067

§ 371 Date: Jan. 6, 2022

Related U.S. Application Data
(60) Provisional application No. 62/871,557, filed on Jul. 8, 2019.

(51) Int. Cl. G01N 33/543; G01N 33/58; G01N 33/569; G01N 33/76

(52) U.S. Cl. CPC G01N 33/54388; G01N 33/587; G01N 2333/165; G01N 33/76; G01N 33/56983

(57) ABSTRACT

Lateral flow immunoassay devices, systems, methods, and kits described herein identify, measure, detect, and analyze analytes of interest in a sample. The lateral flow' immunoassay devices described herein include a test strip without additional features common to traditional lateral flow7 immunoassays, such as housing, pads, or other materials that require complex manufacturing equipment and protocols. Thus, the devices, systems, methods, and kits described herein relate to simplified devices that eliminate complex and expensive manufacture equipment and methods.

### CROSS-REFERENCE TO RELATED APPLICATIONS

This application claims the benefit of U.S. Provisional Application No. 62/871,557, filed Jul. 8, 2019, which is hereby incorporated by reference in its entirety.

### FIELD

Embodiments of the present invention relate to devices, methods, and kits for identifying, measuring, detecting, or analyzing analytes of interest in a sample. In particular, the systems, devices, and kits include simplified lateral flow assays that include treated printer paper and a backing card. Additional embodiments relate to methods of making the simplified lateral flow assays, and methods of using the simplified lateral flow assays for identifying, measuring, detecting, or analyzing analytes of interest in a sample, such as a biological sample from a subject.

### BACKGROUND

Throughout the world, individuals struggle to overcome or live with health challenges, which are wide and varied. Healthy crises, including infections diseases, obesity, cardiac disease, diabetes, neurological disease, and pandemics inflict millions worldwide. Often, early diagnosis of such health challenges can reduce the strain on healthcare, improve patient outlook, and reduce the spread of such diseases.

For illustration, according to the World Health Organization, the number of patients with diabetes has risen to 422 million, with 3.7 million deaths caused by high glucose per year. The World Health Report indicates that 50,000 people die of infectious diseases daily, many of which could be prevented or cured. Approximately 76,000 women and 500,000 babies die worldwide from preeclampsia and hypertensive disorders annually. In developing countries, women are seven times more likely to develop preeclampsia than women in developed countries. The World Allergy Organization estimated in 2011 that 30-40% of the world's population experiences an allergy to one or more allergens. The novel coronavirus first isolated in Wuhan China, had more infections than the previous sever acute respiratory syndrome (SARS) outbreak of 2002 and 2003, and results in coronavirus disease 2019 (COVID-19). The most severe form of the infection is Severe Acute Respiratory Syndrome Coronavirus 2 (SARS-CoV-2), characterized as acute respiratory compromise followed by acute respiratory failure, with relatively high morbidity and mortality secondary to irreversible pulmonary tissue injury, despite intense supportive medical care. These examples illustrate that in a world of chronic disease, infectious agents, allergies, intolerances, and health complications, the need for diagnostic tests is becoming ever greater.

Lateral flow immunoassays (LFIs) are simple diagnostic tests for the early detection of a variety of diseases or disorders. Advantages of LFIs include equipment-free assays that produce results in short periods of time, and typically require small sample volumes. LFIs are easy to run, with straightforward results. Despite these advantages in traditional LFIs, additional challenges remain. For example, although traditional LFIs are accessible and affordable in wealthy regions or in laboratory or clinical settings, traditional LFIs are less accessible in impoverished regions or in field settings.

In any setting, diagnostic assays must be non-invasive and deliver quick results at the point of care (POC). In low-resource settings, a clinic might be without advanced training, equipment, or electricity, so it is critical that tests are easy to understand, run, and interpret. To reduce costs in low-resource settings, the tests also need to be easy, straightforward, and inexpensive to assemble; lowering the manufacturing costs reduces the cost of the test. To manufacture traditional LET tests, a well-equipped lab would have a striper (a machine that immobilizes antibodies to nitrocellulose membrane), a laminator (a machine that aids the assembly of the layered lateral flow cards), and a test shear (a machine that cuts laminated cards into individual tests). Additional materials required to develop traditional LFIs include nitrocellulose membrane, plastic backing cards, conjugate, sample and absorbance pads. Costs for striper, laminators, shears, and LFI materials limit access to LFI production.

For small team operations, laboratories or clinics in low-resource settings, or in field groups without access to the equipment or materials, access to traditional LFI is limited or entirely out of reach.

### SUMMARY

Embodiments of the systems, devices, methods, and kits provided herein relate to improved lateral flow immunoassays (LFIs) that can be readily manufactured without the requirement for expensive or inaccessible equipment and materials. Embodiments of the improved LFIs disclosed herein are inexpensive to manufacture, provide accurate and rapid test results, and may be manufactured in low cost settings, in the field, and at the point of care (POC) to provide rapid POC diagnostics.

Some aspects provide for a test strip. In some embodiments, the test strip comprise a flow path configured to receive a fluid sample, wherein the flow path comprises activated substrate that has been blocked with a blocking agent and a test line coupled to the flow path, wherein the test line comprises immobilized test agent specific to an analyte of interest.

In some embodiments, the activated substrate is aldehyde functionalized paper. In some embodiments, the activated substrate is activated with potassium periodate. In some embodiments, the blocking agent is bovine serum albumin, casein, or a solution of powdered milk. In some embodiments, the test agent is an antibody or a protein specific to the analyte of interest. In some embodiments, the flow path is configured to receive a fluid sample comprising labeled analyte of interest, wherein the labeled analyte of interest is labeled with a detectable label. In some embodiments, the test strip further comprises control line coupled to the flow path, wherein the control line comprises immobilized control agent specific to a detectable label. In some embodiments, the detectable label comprises a metal nanoparticle conjugated to an antibody specific to the analyte of interest. In some embodiments, the metal nanoparticle is a gold nanoparticle. In some embodiments, the test strip further comprises a backing card. In some embodiments, the fluid sample is selected from the group consisting of a blood, plasma, urine, sweat, nasal, lacrimal, or saliva sample.

Some aspects provide for a kit. In some embodiments, the kit comprises a substrate, an activation reagent, a dispensing device comprising test agent specific to an analyte of interest, a blocking reagent, and a detectable label specific to the analyte of interest. In some embodiments, the substrate is printer paper, the activation reagent is potassium periodate, the dispensing device is a rollerball pen comprising the test agent, the test agent is an antibody or protein specific to the analyte of interest, the blocking reagent is bovine serum albumin, casein, or a solution of powdered milk, and the detectable label is a gold nanoparticle conjugated to an antibody specific to the analyst of interest. In some embodiments, the kit further comprises a control dispensing device comprising a control agent. In some embodiments, the kit further comprises a backing card.

Some aspects provides a method of manufacturing a test strip. In some embodiments, the method of manufacturing a test strip comprises contacting a substrate with an activation reagent to generate activated substrate, contacting the activated substrate with a test agent at a test line; and contacting the activated substrate after contacting with a blocking reagent. In some embodiments, the contacting the activated substrate with the test agent comprises applying the test agent to the activated substrate using a dispensing device. In some embodiments, the dispensing device is a pen or marker comprising the test agent. In some embodiments, the dispensing device is a rollerball pen. In some embodiments, the activation reagent is potassium periodate. In some embodiments, test agent comprises an antibody or protein specific to the analyte of interest. In some embodiments, the blocking reagent comprises bovine serum albumin, casein, or a solution of powdered milk. In some embodiments the method of manufacturing a test strip, further comprises contacting the activated substrate after contacting the activated substrate with a test agent at a test line with a control agent at a control line. In some embodiments, the control agent comprises an antibody or protein specific to a detectable label. In some embodiments, the detectable label comprises a metal nanoparticle conjugated to an antibody specific to the analyte of interest. In some embodiments, the metal nanoparticle is a gold nanoparticle. In some embodiments, the substrate is paper.

In some embodiments, the method of manufacturing a test strip comprises soaking paper in 0.03 M potassium periodate to generate activated paper, applying an antibody or protein specific to analyte of interest having a concentration of about 1 mg/mL to the activated paper using a first rollerball pen, applying an antibody or protein specific to a detectable label having a concentration of about 1 mg/mL to the activated paper using a second rollerball pen, and soaking the activated paper in a solution of 5% powdered milk. In some embodiments, the method of manufacturing a test strip further comprises applying a backing card to the activated substrate, and cutting the activated substrate into test strips.

Some aspects provide for a method for measuring an analyte in a fluid sample. In some embodiments, the method comprises providing a fluid sample having or suspected of having an analyte of interest, contacting the fluid sample with a detectable label that specifically binds analyte of interest, wherein the detectable label binds analyte of interest in the fluid sample to form a labeled analyte of interest, contacting a test strip with a sample, wherein the test strip comprises: a flow path configured to receive a fluid sample, wherein the flow path comprises activated substrate that has been blocked with a blocking agent, and a test line coupled to the flow path, and comprising immobilized test agent specific to an analyte of interest, flowing the sample through the test strip, binding the labeled analyte of interest to the immobilized test agent at the test line, and detecting a signal from the labeled analyte of interest bound to the immobilized test agent at the test line.

In some embodiments, the method for measuring an analyte in a fluid sample detection signal is an optical signal. In some embodiments, the analyte of interest is a protein or a viral particle. In some embodiments, the detectable label comprises a metal nanoparticle conjugated to an antibody that specifically binds analyte of interest. In some embodiments, the metal nanoparticle is a gold nanoparticle. In some embodiments, the sample is selected from the group consisting of a blood, plasma, urine, sweat, nasal, lacrimal, or saliva sample. In some embodiments, the method for measuring an analyte further comprises comparing an intensity of the signal at the test line to an intensity of a control signal of known concentrations of analyte of interest. In some embodiments, the method for measuring an analyte further comprises increasing an intensity of the signal at the test line by incubating the test strip in a signal enhancing solution. In some embodiments, the signal enhancing solution is a solution of silver.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments, and are not intended to be limiting in scope.

FIG. 1A illustrates an exemplary traditional lateral flow immunoassay. FIG. 1B illustrates an exemplary simplified empowering lateral flow immunoassay.

FIG. 2 depicts the mechanism for aldehyde functionalization on cellulose (panel A) and protein binding to aldehyde functionalized cellulose (panel B).

FIG. 3 depicts an exemplary method for measuring an analyte of interest using the activated substrates described herein.

FIGS. 4A and 4B depict exemplary test results for human chorionic gonadotropin (hCG) using the activated substrates described herein. FIG. 4A depicts a negative control, with no hCG present in the sample. FIG. 4B depicts a positive test result, with hCG present in the sample. The results are shown in triplicate, with the fourth strip shown as a commercially available strip for comparison (far right).

### DETAILED DESCRIPTION

Although the invention is described in various exemplary embodiments and implementations as provided herein, it should be understood that the various features, aspects, and functionality described in one or more of the individual embodiments are not limited in their applicability to the particular embodiment with which they are described. Instead, they can be applied alone or in various combinations to one or more of the other embodiments of the invention, whether the embodiments are described or whether the features are presented as being a part of the described embodiment. The breadth and scope of the present invention should not be limited by any exemplary embodiments described or shown herein.

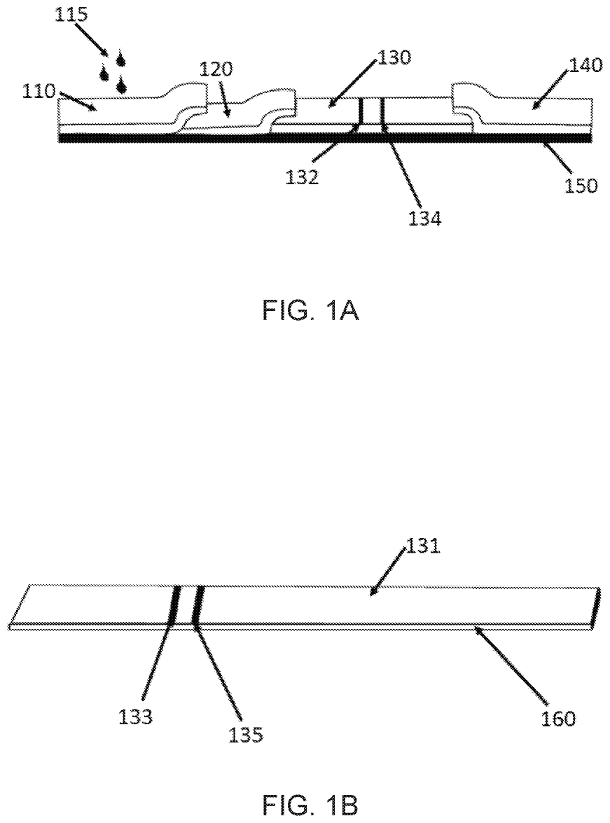

Traditional lateral flow immunoassays (LFIs) require expensive raw materials, and expensive manufacturing equipment. As shown in FIG. 1A, traditional LFIs include a nitrocellulose substrate 130 that acts as a test strip, and which is conjugated to multiple additional components, including a sample pad 110, a conjugate pad 120 an absorbent pad 140, and a backing 150. Not only is the material required for traditional LFIs expensive, but it also requires expensive manufacturing processes, including combining these elements to form the traditional LFI. Additional components often include housing and complex combination of reagents and materials. In addition, traditional LFIs require expensive equipment for manufacturing the LFI itself, and added equipment for striping antibody in a test line 132 and in a control line 134. Thus, traditional LFIs are incapable of being manufactured or assembled onsite. These requirements for materials and manufacturing processes for traditional LFIs increases costs, limits accessibility, and limits applicability of traditional LFIs.

Embodiments provided herein relate to devices, methods, and kits for improved and simplified LFIs. Embodiments of the devices disclosed herein provide simplified LFIs that overcome limitations of traditional LFIs, thereby empowering the use of the simplified LFIs by increasing accessibility and applicability, and concomitantly decreasing costs. The simple empowering LFIs (seLFIs) described herein provide several advantages over existing LFIs. For example, the seLFIs discloses herein may be manufacture on site of performance of an assay, and without the need for complicated or expensive manufacturing equipment. Thus, so long as a few materials are available, including binding agents against the analyte of interest, standard paper, and an activation reagent, the seLFI disclosed herein may be quickly, inexpensively, and onsite manufactured. Thus, the seLFI devices described herein do not require striping machinery for placement of test and/or control lines. Instead, antibodies are deposited onto functionalized paper using an antibody pen. Because equipment is not used, a related advantage is the elimination of a power source in order to make the seLFI devices disclosed herein. Because the paper is functionalized using an activation reagent, the functionalized paper is more hydrophilic than nitrocellulose paper used in traditional LFIs, thereby resulting in increased rapidity of assay development and results. Another advantage is the use of the antibody pen to place antibodies onto the seLFI. The antibody pen is inexpensive, portable, and does not require electricity. The antibodies may be lyophilized and stored separately or in the pen, and only the addition of water is needed, thus creating a pen that can be stored for extended periods of time. A related advantage includes the minimal requirements for materials. Thus, whereas traditional LFIs require multiple pads, including sample pads, conjugate pads, and absorbance pads, in addition to a detection membrane, the seLFI disclosed herein requires only a single sheet of paper that is functionalized using an activation reagent. Thus, the manufacture is significantly simplified by removing attachment of pads to a test strip. An advantage of on-site production of the seLFI test was exemplified during the COVID19 pandemic when supply lines for all LFI components were disrupted and in short, or non-existent supply as LFI companies developed and produced LFI tests for COVID19, which stopped availability of supplies for other LFI tests. Additional pads or components can be added to the seLFI for filtration, for example, but are not required in the devices disclosed herein. An additional advantage includes the elimination of required buffers to run the assay. Additional advantages include long-term storage of the seLFI assays, where test results remain accurate for extended periods of time. These and other advantages results in a test device that can be made and used on site without complicated equipment and/or materials.

### Test Strips

As shown in FIG. 1B, a seLFI includes a substrate 131 that has been activated. In some embodiments, the activated substrate is referred to herein as a test strip. In some embodiments, the seLFI further includes a backing 160, which may comprise a plastic or other firm material. In some embodiments, the substrate 131 is any type of hydrophilic medium that is capable of being activated. In some embodiments, the substrate is any type of hydrophilic medium that is readily available, that is low cost, or that otherwise may be used in a field setting without a requirement for specialized equipment. In some embodiments the hydrophilic medium is paper, such as printer paper or other readily available paper. In some embodiments, the substrate is activated with an activating agent. In some embodiments, the activating agent includes a periodate. As used herein, the term “periodate” includes periodate or periodic acid, can include a metaperiodate or orthoperiodate, and may include various salts of periodate, such as sodium periodate (NaIO₄) or potassium periodate (KIO₄). In some embodiments, activation with periodate results in an aldehyde-functionalized substrate. Without wishing to be bound by theory, such activation creates aldehyde groups that can form Schiff bases with proteins that contact the substrate, thereby immobilizing the proteins. In some embodiments, activation may include nitration of the substrate, for example, by contacting the substrate with sulfuric acid and nitric acid. Other means for activation the substrate may be employed, wherein the activated substrate is capable of binding proteins.

In some embodiments, as shown in FIG. 1B, the test strip includes a flow path that includes a test line 133. In some embodiments, the test strip includes a control line 135, or one or more additional test or control lines. In some embodiments, the test line 133 may be formed on the activated substrate 131 by contacting the activated substrate 131 with a test agent that is specific to an analyte of interest. In some embodiments, a control line 135 may be formed on the activated substrate 131 by contacting the activated substrate 131 with a control agent that is specific to a detectable label. In some embodiments, the test agent is an antibody or protein that specifically binds an analyte of interest. In some embodiments, the control agent is an antibody or protein that specifically binds a detectable label. In some embodiments, a detectable label includes a metal nanoparticle that is conjugated to an antibody that specifically binds to the analyte of interest. In some embodiments, the metal nanoparticle is a nanoparticle of gold, silver, magnesium, zinc, calcium, manganese, copper, palladium, nickel, platinum, titanium, cerium, iron, thallium, molybdenum, or an alloy, oxide, hydroxide, sulfide, nitrate, phosphate, fluoride, or chloride thereof. In some embodiments, the metal nanoparticle emits a detectable optical signal.

Although embodiments of the present disclosure are described herein by reference to an “optical” signal, it will be understood that assays described herein can use any appropriate material for a label in order to generate a detectable signal, including but not limited to fluorescence-type latex bead labels that generate fluorescence signals and magnetic nanoparticle labels that generate signals indicating a change in magnetic fields associated with the assay. Labels can take many different forms, including a molecule or composition bound or capable of being bound to an antibody that is detectable by spectroscopic, photochemical, biochemical, immunochemical, electrical, optical, visual, or chemical means. Examples of labels include enzymes, colloidal gold particles (also referred to as gold nanoparticles or AuNPs), colored latex particles, radioactive isotopes, co-factors, ligands, chemiluminescent or fluorescent agents, protein-adsorbed silver particles, protein-adsorbed iron particles, protein-adsorbed copper particles, protein-adsorbed selenium particles, protein-adsorbed sulfur particles, protein-adsorbed tellurium particles, protein-adsorbed carbon particles, and protein-coupled dye sacs. In some embodiments, conjugating a label to an antibody may be performed passively or covalently, depending on the application and the skill of the end user. In some embodiments, the antibodies are purchased with a label.

In some embodiments, the analyte of interest is any analyte to be detected for diagnostic or analytical purposes. Thus, as used herein, “analyte” refers to a substance to be detected. For instance, analytes may include antigenic substances, haptens, antibodies, and combinations thereof. Analytes include, but are not limited to, toxins, organic compounds, proteins, peptides, microorganisms, amino acids, nucleic acids, hormones, steroids, vitamins, drugs (including those administered for therapeutic purposes as well as those administered for illicit purposes), drug intermediaries or byproducts, bacteria, virus particles, and metabolites of or antibodies to any of the above substances. Specific examples of some analytes include ferritin; hepcidin; creatinine kinase MB (CK-MB); human chorionic gonadotropin (hCG); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein (CRP); lipocalins; IgE antibodies; cytokines; TNF-related apoptosis-inducing ligand (TRAIL); vitamin B2 micro-globulin; interferon gamma-induced protein 10 (IP-10); glycated hemoglobin (Gly Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Additional analytes include viral particles or antibodies to viruses, including hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; SARS virus, particles, or antibodies against such (including SARS-CoV-2); MERS; or other viruses. For example, the devices disclosed herein may be used to detect SARS-CoV-2 spike protein or SARS-CoV-2 nucleocapsid protein (N-protein). Any analyte now known are discovered to be known to be associated with a particular disease, disorder, or condition, and which can be bound using a binding partner immobilized on a substrate may be an analyte of interest. Additional analytes may be included for purposes of biological or environmental substances of interest.

The analyte of interest may be based on the particular disease, disorder, or condition to be tested in an individual. The disease, disorder, or condition could be any disease, disorder, or condition that has or is suspected of having an analyte at levels that are distinguishable from a healthy state. In some embodiments, the disease, disorder, or condition is diabetes, preeclampsia, hypertension, kidney disease, anemia, infectious diseases, malaria, viral infection, bacterial infection, neurological diseases or disorders, or any other diseases, disorders, or conditions that has or that is suspected of having altered levels of analyte. In some embodiments, the test strips described herein are used for evaluating nutritional status, detecting causes of allergies, or in the aid of general health safety diagnostics.

As used herein, the term “viral infection” has its ordinary meaning as understood in light of the specification, and refers to an infection of a subject by a virus. As used herein, the term “virus” has its ordinary meaning as understood in light of the specification, and refers to obligate intracellular parasites of living but noncellular nature, consisting of DNA or RNA and a protein coat. Viruses range in diameter from about 20 to about 300 nm. Class I viruses (Baltimore classification) have a double-stranded DNA as their genome (such as Adenoviruses, Herpesviruses, or Poxviruses); Class II viruses have a single-stranded DNA as their genome (such as Parvoviruses); Class III viruses have a double-stranded RNA as their genome (such as Reoviruses); Class IV viruses have a positive single-stranded RNA as their genome, the genome itself acting as mRNA (such as Picornaviruses or Togaviruses); Class V viruses have a negative single-stranded RNA as their genome used as a template for mRNA synthesis (such as Orthomyxoviruses or Rhabdoviruses); Class VI viruses have a positive single-stranded RNA genome but with a DNA intermediate not only in replication but also in mRNA synthesis (such as Retroviruses); and Class VII viruses have a double-stranded DNA genome but with an RNA intermediate in life-cycle (such as Hepadnaviruses). The majority of viruses are recognized by the diseases they cause in plants, animals and prokaryotes. Viruses of prokaryotes are known as bacteriophages.

In some embodiments, the virus is a DNA virus. DNA viruses include, but are not limited to a virus belonging to one of the following families: adenovirus, astrovirus, hepadnavirus, herpesvirus, papovavirus, and poxvirus. In other embodiments, the virus is an RNA virus. RNA viruses include but are not limited to a virus belonging to one the following families: arenavirus, bunyavirus, calcivirus, coronavirus, filovirus, flavivirus, orthomyxovirus, paramyxovirus, picornavirus, reovirus, retrovirus, rhabdovirus, or togavirus.

The individual may be an animal, a mammal, and a human. A sample obtained from a subject can include any fluid from the subject that may contain an analyte of interest. In some embodiments, the sample is blood, plasma, urine, sweat, nasal, lacrimal, or saliva sample.

Contacting the substrate 131 with a test agent for the test line 133 or with a control agent for the control line 135 may be achieved by applying the test agent and/or the control agent to the activated substrate using a dispensing device. A dispensing device, as used herein, is any type of device capable of dispensing in a precise, intention, or controlled manner a volume of test agent and/or control agent to the activated substrate. In some embodiments, the dispensing device is a syringe, a pen, a marker, or any other device capable of dispensing the test agent and/or the control agent. In some embodiments, the dispensing device is a pen, a felt tip pen, a brush pen, a paint pent, a fountain pen, a regular pen, or a rollerball pen. In some embodiments, the pen is a pen that is purchased without any ink. In some embodiments, the pen is a pen that is purchased with ink, but wherein the ink is removed from the pen, and wherein the pen is thoroughly washed and cleaned to remove all ink residue. The dispensing device, including a pen, may be filled with the test agent or the control agent. In some embodiments, the test agent or the control agent is prepared in a solution to generate an antibody ink. As used herein, the term “antibody ink” refers to a test agent or control agent that is prepared in a solution and deposited into a dispensing device, such as a pen. In some embodiments, the viscosity of the antibody ink is altered by adding a viscous material, such as glycerol. In some embodiments, a rollerball pen is filled with an antibody ink that includes a test agent, and a test line is drawn on the activated substrate by a user. In some embodiments, a rollerball pen is filled with an antibody ink that includes a control agent, and a control line is drawn on the activated substrate by a user. Additional dispensing devices may be prepared to generate one or more additional test or control lines having agents specific to an analyte of interest, or to provide one or more control zones. The test and control lines may be drawn in a straight line using the dispensing device by using a straight edge. Upon application of the antibody ink to the substrate, the antibody or protein contained in the antibody ink is immobilized to the substrate.

The concentration of antibody or protein in the antibody ink may be any suitable concentration for capture of an analyte of interest or capture of the detectable label. For example, the concentration of the antibody or protein may be in an amount of about 0.0001 mg,/mL to about 10 mg/mL, such as 0.0001, 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9 or 10 mg/mL, or at a concentration within a range defined by any two of the aforementioned values. In some embodiments, the volume of antibody ink deposited on the activated substrate is dependent on the size of the activated substrate, and the desired size of the test or control lines. In some embodiments, the volume of antibody ink deposited on the activate substrate is in an amount of about 0.1 μL to about 20 μL, such as 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 μL or in an amount within a range defined by any two of the aforementioned values.

As exemplified in the embodiments described herein, the use of readily available substrates, which may be readily activated using an activation agent, and then manually adding a test line and/or a control line increases the accessibility, applicability, and ease of making and using the seLFI devices described herein, and also decreases the complexity and cost of manufacture and use compared to traditional LFIs. Thus, the devices disclosed herein (and referred to herein as test strips, activated substrates, or seLFIs) may be both manufactured and used in low resource settings, in the field, at point of care (POC), at home, or in environments without power sources in a variety of applications depending on the biomarker of interest.

Due to the availability of the materials required for the assays described herein, the materials are also less prone to limitations of supply chain that occur during global pandemics or shortages, thereby enabling the continued manufacture and use of the devices even in time of global crises or supply chain issues.

### Kits

Embodiments provided herein relate to kits. In some embodiments, a kit is provided that enable a user to manufacture and use a seLFI on site and without the need for specialized equipment or a power supply. In some embodiments, the kit includes a substrate, an activation agent, a dispensing device, and a blocking agent. In some embodiments, the kit further includes a test agent that can be inserted into the dispensing device, or a user can obtain a test agent from a different source. In some embodiments, the kit further includes a detectable label, or components of a detectable label, including metal nanoparticles and an antibody against the analyte of interest, or the user can obtain the detectable label or components thereof from a different source. Thus, in some embodiments, the kit includes a substrate, an activation agent, a dispensing device, a test agent, a blocking agent, and a detectable label. Components included in the kit, including a substrate, an activation agent, a dispensing device, a test agent, a blocking agent, and a detectable label are described herein elsewhere. In some embodiments, the kit further includes a control agent. In some embodiments, the kit further includes a backing card.

In some embodiments, the kit further includes a manual or instructions for manufacturing the test strips, including activating the substrate, forming a test and/or control line on the activated substrate, blocking the activated substrate, forming labeled analyte of interest, and/or performing an assay on the manufactured seLFI device. In some embodiments, the kit further includes a manual or instruction for analyzing results obtained from using the manufactured seLFI device. Instructions may include interpretation of a detectable signal for qualification and/or quantification of an analyte of interest in a sample. In some embodiments, the instructions are provided in print format or in electronic format, such as video, audio, mobile device application, or online format. For example, such electronic formats may be provided in a physical device, such as a disc or other accessible electronic medium, or may be accessed by scanning a quick response (QR) code to obtain online or application access.

### Methods of Manufacture

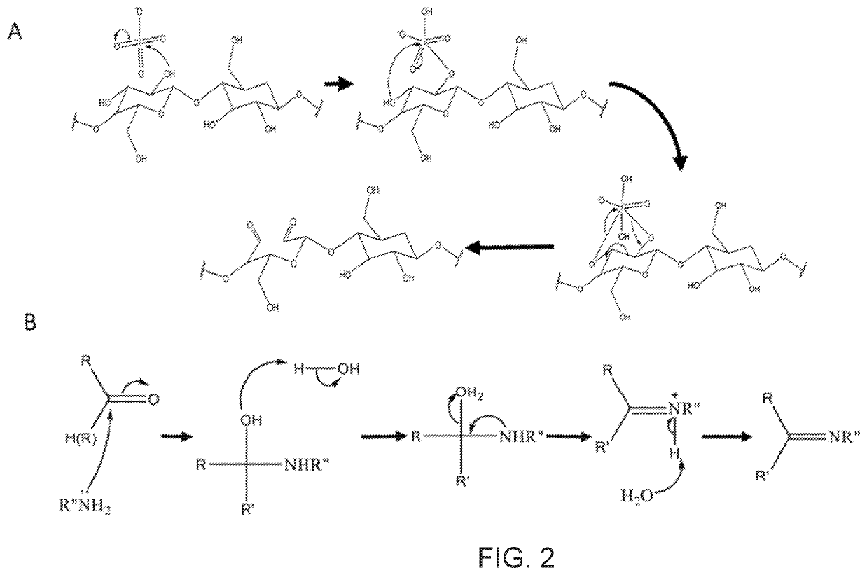

Embodiments provided herein relate to methods for making the seLFI devices described herein. In some embodiments, the methods include providing a substrate that is capable of being activated for immobilization of a test agent. In some embodiments, the substrate is any type of hydrophilic medium that is capable of being activated. In some embodiments, the substrate is any type of hydrophilic medium that is readily available, that is low cost, or that otherwise may be used in a field setting without a requirement for specialized equipment. In some embodiments, the hydrophilic medium is paper, such as printer paper or other readily available paper. Some embodiments provided herein relate to methods of activating the substrate for immobilization of a test agent to a specified location on the substrate. In some embodiments, activating the substrate include contacting the substrate with an activation reagent to generate an activated substrate. In some embodiments, the activating agent is a periodate. In some embodiments, the activation reagent is sodium periodate (NaIO₄) or potassium periodate (KIO₄). In some embodiments, activation with periodate results in an aldehyde-functionalized substrate. As shown in FIG. 2, activation generates aldehydes on cellulose. Panel A of FIG. 2 shows the process of aldehyde functionalization on cellulose. Panel B of FIG. 2 shows the process of protein binding to aldehyde functionalized paper, which is the process of placing a test agent or control agent to the activated substrate.

In some embodiments, the activation reagent is added in an amount ranging from about 0.001 M to about 1 M, such as 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 M, or in a concentration within a range defined by any two of the aforementioned values. In some embodiments, the substrate is soaked, rinsed, sprayed, or otherwise contacted with a solution of activation reagent. In some embodiments, the contacting is performed for a time period ranging from a few seconds, to a few hours, for example 1, 2, 3, 4, 5, 6, 7, 8, 9, 10, 15, 30, 45, or 60 seconds, or 1, 2, 3, 4, 5, 6, 7, 8, 9, 10, 15, 30, 45, or 60 minutes, or 1, 2, 3, 4, 5, 6, 7, 8, 9, 10, 11, or 12 hours, or for a period of time within a range defined by any two of the aforementioned values. In some embodiments, the contacting is performed at a temperature sufficient to activate the substrate, such as at a temperature ranging from about 15° C. to about 80° C., such as 15, 20, 25, 30, 35, 40, 45, 50, 55, 60, 65, 70, 75, or 80° C., or at a temperature within a range defined by any two of the aforementioned values. In some embodiments, the substrate is removed from the activation reagent, and rinsed one or more times in water by contacting the activated substrate with water for a period of time, such as for a few seconds or a few minutes. In some embodiments, rinsing in water is performed one, two, three, four, five, six or more times. In some embodiments, following rinsing, the activated substrate is dried for a minimum of a few minutes to a few hours, such as 1, 2, 3, 4, 5, 6, 7, 8, 9, 10, 15, 30, 45, or 60 minutes, or 1, 2, 3, 4, 5, 6, 7, 8, 9, 10, 11, or 12 hours or more, or for a period of time within a range defined by any two of the aforementioned values. In some embodiments, the activated substrate is dried at a temperature ranging from about 15° C. to about 80° C., such as 15, 20, 25, 30, 35, 40, 45, 50, 55, 60, 65, 70, 75, or 80° C., or at a temperature within a range defined by any two of the aforementioned values. Drying can be performed in an incubator or in the open environment.

Some embodiments provided herein relate to generating a test line on the activated substrate. In some embodiments, a control line is also added to the activated substrate. In some embodiments, the test and/or control lines are added after the activated substrate has been dried. In some embodiments, a test line is generated by applying test reagent to the activated substrate. In some embodiments, a control line is generated by applying control reagent to the activated substrate. In some embodiments, the test line and the control line are placed on the activated substrate at a sufficient distance from one another to be able to clearly distinguish one from another. Thus, in some embodiments, the test line and the control line are at least 0.1 mm apart, such as 0.1, 0.5, 1, 2, 3, 4, 5, 6, 7, 8, 9, or 10 mm apart, or a length within a range defined by any two of the aforementioned values. In some embodiments, the test line and the control line are placed on the activated substrate within one third of the total length of the activated substrate, such as a lower third region of the activated substrate.

In some embodiments, the test agent for generating the test line, and the control agent for generating the control line are applied at a concentration of about 0.0001 mg/mL to about 10 mg/mL, such as 0.0001, 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9 or 10 mg/mL, or at a concentration within a range defined by any two of the aforementioned values. In some embodiments, the volume of test or control agent (sometimes referred to herein as antibody ink) that is deposited on the activated substrate is dependent on the size of the activated substrate, and the desired size of the test or control lines. Additional factors in determining the concentration and amount of deposited antibody ink could include the affinity of the test or control reagent for their binding partners. In some embodiments, the volume of antibody ink deposited on the activate substrate is in an amount of about 0.1 μL to about 20 μL, such as 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 μL or in an amount within a range defined by any two of the aforementioned values.

In some embodiments, depositing antibody ink on the activated substrate is performed using a dispensing device. In some embodiments, a dispensing device is a syringe, a pen, a marker, or any other device capable of dispensing the test agent and/or the control agent. In some embodiments, the dispensing device is a pen, a felt tip pen, a brush pen, a paint pent, a fountain pen, a regular pen, or a rollerball pen. In some embodiments, the pen is a pen that is purchased without any ink. In some embodiments, the pen is a pen that is purchased with ink, but wherein the ink is removed from the pen, and wherein the pen is thoroughly washed and cleaned to remove all ink residue. The dispensing device, including a pen, may be filled with the test agent or the control agent. In some embodiments, the test agent or the control agent is prepared in a solution to generate an antibody ink. As used herein, the term “antibody ink” refers to a test agent or control agent that is prepared in a solution and deposited into a dispensing device, such as a pen. In some embodiments, the viscosity of the antibody ink is altered by adding a viscous material, such as glycerol. In some embodiments, a rollerball pen is filled with an antibody ink that includes a test agent, and a test line is drawn on the activated substrate by a user. In some embodiments, a rollerball pen is filled with an antibody ink that includes a control agent, and a control line is drawn on the activated substrate by a user. Additional dispensing devices may be prepared to generate one or more additional test or control lines having agents specific to an analyte of interest, or to provide one or more control zones. The test and control lines may be drawn in a straight line using the dispensing device by using a straight edge. Upon application of the antibody ink to the substrate, the antibody or protein contained in the antibody ink is immobilized to the substrate. In some embodiments, the antibody pen does not exclude the use of putting the antibody ink in an ink jet printer for delivery to the seLFI test strip to create the test and control lines or placing the antibody ink into a striping machine used for traditional LFI production to deliver the antibodies to the test strip.

In some embodiments, activation may include nitration of the substrate, for example, by contacting the substrate with sulfuric acid and nitric acid. Other means for activation the substrate may be employed, wherein the activated substrate is capable of binding proteins.

Following activation of the substrate and placing the test and control lines on the activated substrate, some embodiments provided herein relate to blocking the activated substrate. In some embodiments, blocking the activated substrate is performed to prevent non-specific binding to regions other than the test and control lines. In some embodiments, blocking is performed by contacting the activated substrate with a blocking reagent. In some embodiments, the blocking reagent is bovine serum albumin (BSA), casein, or a solution of powdered milk. Other blocking reagents could include any available proteins that may be abundant at the site of manufacture, such as, for example, proteins ground up and dissolved from a local plant, such as a bean. The concentration of blocking reagent can be any concentration capable of blocking the activate substrate. For example, blocking can include a concentration of BSA or casein in an amount of about 0.0001 mg/mL to about 10 mg/mL, such as 0.0001, 0.0005, 0.001, 0.005, 0.01, 0.05, 0.1, 0.2, 0.3, 0.4, 0.5, 0.6, 0.7, 0.8, 0.9, 1, 2, 3, 4, 5, 6, 7, 8, 9 or 10 mg/mL, or at a concentration within a range defined by any two of the aforementioned values. In some embodiments, blocking includes contacting the activated substrate with a solution of powdered milk, wherein the milk is present in an amount of about 0.5% w/v to about 15% w/v, such as 0.5, 1, 2, 3, 4, 5, 6, 7, 8, 9, 10, 11, 12, 13, 14, or 15% w/v powdered milk, or in an amount within a range defined by any two of the aforementioned values. Following the blocking step, the activated substrate is allowed to dry.

In some embodiments, after the activated substrate is blocked and dried, the activated substrate is attached to a backing card. In some embodiments, after the activated substrate is blocked and dried, the activated substrate is cut into strips. In some embodiments, the strips are cut to a length of at least 10 mm, such as 10, 15, 20, 25, 30, 35, 40, 45, 50, 55, 60, 65, 70, 75, 80, 85, 90, 95, or 100 mm in length, or a length within a range defined by any two of the aforementioned values. In some embodiments, the strips are cut to a width of about 1 to about 10 mm, such as 1, 2, 3, 4, 5, 6, 7, 8, 9, or 10 mm, or a width within a range defined by any two of the aforementioned values. In some embodiments, the length and width of the substrate is cut to a length and width larger than these values, or any length and/or width desirable for the given application.

In some embodiments, the methods of manufacturing the test strip includes manufacturing a single test strip. In some embodiments, the methods include manufacturing sheets of substrate that may later be cut into multiple test strips, thereby enabling mass manufacture of multiple test strips in a single manufacturing process.

### Methods of Use

Embodiments provided herein relate to methods of using the seLFI devices described herein for detecting an analyte of interest. In some embodiments, a test strip as described herein is provided. In some embodiments, the test strip is contacted with a sample having or suspected of having an analyte of interest. In some embodiments, the sample is a any fluid from the subject that may contain an analyte of interest. In some embodiments, the sample is a blood, a plasma, a urine, a sweat, a nasal, a lacrimal, or a saliva sample.

In some embodiments, the analyte is ferritin; hepcidin; creatinine kinase MB (CK-MB); human chorionic gonadotropin (hCG); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein (CRP); lipocalins; IgE antibodies; cytokines; TNF-related apoptosis-inducing ligand (TRAIL); vitamin B2 micro-globulin; interferon gamma-induced protein 10 (IP-10); glycated hemoglobin (Gly Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Additional analytes include viral particles or antibodies to viruses, including hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; SARS virus, particles, or antibodies against such (including SARS-CoV-2); MERS; or other viruses. Additional analytes may be included for purposes of biological or environmental substances of interest.

In some embodiments, the sample is contacted with a detectable label. In some embodiments, the detectable label is a metal nanoparticle conjugated to a binding agent that specifically binds an analyte of interest. In some embodiments, the metal nanoparticle is a nanoparticle of gold, silver, magnesium, zinc, calcium, manganese, copper, palladium, nickel, platinum, titanium, cerium, iron, thallium, molybdenum, or an alloy, oxide, hydroxide, sulfide, nitrate, phosphate, fluoride, or chloride thereof. In some embodiments, the metal nanoparticle emits a detectable optical signal. In some embodiments, the binding agent is an antibody or protein that specifically binds the analyte of interest. For example, a detectable label can be gold nanoparticle conjugated to anti-analyte antibody. In some embodiments, the detectable label is added to the sample in an amount such that excess detectable label is present, such that all analyte of interest is labeled with the detectable label, and free detectable label remains. In some embodiments, detectable label is added in an amount of about 0.1 μL to about 20 μL such as 0.1, 0.5, 1, 2, 3, 4, 5, 6, 7, 8, 9, 10, 15, or 20 μL or an amount within a range defined by any two of the aforementioned values of an antibody concentration ranging from about 0.01 to about 10 mg/mL, such as 0.01, 0.05, 0.1, 0.2, 0.3, 0.4, 0.5, 0.6, 0.7, 0.8, 0.9, 1, 2, 3, 4, 5, 6, 7, 8, 9, or 10 mg/mL of an amount within a range defined by any two of the aforementioned values.

In some embodiments, adding detectable label results in formation of labeled analyte of interest in the sample (when analyte of interest is present). In some embodiments, the sample having labeled analyte of interest is contacted with the test strips described herein. In some embodiments, contacting the sample with the test strip causes flow of sample through the test strip due to capillary action. In some embodiments, test agent at the test line captures labeled analyte of interest, resulting in a detectable signal at the test line. In some embodiments, control agent at the control line captures detectable label that is not bound to analyte of interest, resulting in a detectable signal at the control line.

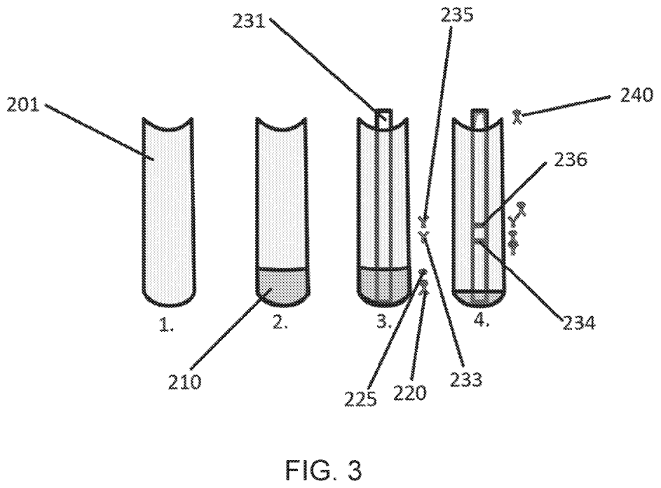

FIG. 3 depicts an exemplary method of performing an assay using the test strips provided herein. As shown in FIG. 3, the method includes providing a test tube 201 (step 1.). To the test tube is added sample 210 having analyte of interest 225 and detectable label 220 (step 2.). In some embodiments, the sample 210 further includes a chase buffer that includes blocking reagent, such as powdered milk. The sample 210 is contacted with the test strip 231 (step 3.). The test strip 231 include a test line having test agent 233 deposited thereon and control agent 235 deposited thereon. After contacting the sample 210 with the test strip 231, sample 210 flows through capillary action through the test strip 231, and labeled analyte binds to the test line 234, providing a detectable signal, indicating that analyte of interest is present in the sample 210 (step 4.). A detectable signal is also emitted at the control line 236, due to excess detectable label 220 present in the sample 210, which is bound by control agent 235. Excess detectable label 240 that does not bind any analyte of interest 225 or does not bind to the control line 236 flows through the test strip 231 and accumulates at the distal end of the test strip, and which may result in a detectable signal indicating accumulation of excess detectable label.

In some embodiments, results of the assay are determined optically, by visual inspection of the test strip. In some embodiments, visual inspection of the signal at the test and/or control line can be performed by comparing the signal intensity to a control test strip having a known quantity of analyte of interest, thereby allowing qualitative and/or quantitative measurement of analyte of interest. In some embodiments, results of the assay are determined using a reader device, which may be capable of determining the intensity of the signal at the test and/or control line. In some embodiments, a reader may include a mobile device, capable of capturing a photograph of the test strip. In some embodiments, the mobile device or other reader device compares the intensity of the signal to a control intensity to determine the quantity of analyte of interest in the sample. Such comparison may be performed, for example, by comparing to known values and known intensities of signal, or by analysis using a mobile application.

Some embodiments provided herein further relate to methods of treatment upon early diagnosis of a disease. For example, a sample from a subject may be obtained from the subject, and subjected to the assays and methods described herein, for example by measuring an analyte of interest in the sample using the test devices described herein. Upon indication of a positive test result (an indication that analyte of interest is present in the sample), a treatment protocol may be administered or provided to the subject. Such treatment may include a combination treatment, or a stand-alone treatment. In some embodiments, the indication of a positive test result may be an indication of an inflammatory disease, heart disease, kidney disease, cancer, Alzheimer's disease, Parkinson's disease, Human Immunodeficiency Virus (HIV), Hepatitis-C virus (HCV), cytomegalovirus (CMV), Dengue virus, Ebola virus, Lassa virus, West Nile virus, rheumatoid arthritis, vasculitis, sarcoid, inflammatory bowel disease, multiple sclerosis, atherosclerosis, diabetes, congestive heart failure, or a combination thereof. The skilled artisan will recognize the treatment protocol that is to be administered or provided to the subject. Such decisions are left for those skilled in the art of treatment.

In some embodiments, a sample from a subject may be obtained from the subject, and subjected to the assays and methods described herein, for example by measuring an analyte of interest in the sample using the test devices described herein. In some embodiments, the method further comprises methods of preventing, treating, or ameliorating at least one symptom of anemia in a subject. In some embodiments, anemia may be anemia of chronic inflammation. In some embodiments, the subject is given synthetic erythropoiesis stimulating agents and iron supplements. In some embodiments, the preventing, treating, or ameliorating at least one symptom of anemia in a subject comprises administering a pharmaceutical composition comprising one or more protease inhibitors. In some embodiments, the one or more protease inhibitors reduces the activity of furin. In some embodiments, the one or more protease inhibitors is selected from the group consisting of amprenavir, atazanavir, boceprevir, darunavir, fosamprenavir, indinavir, lopinavir, nelfinavir, ritonavir, saquinavir, simeprevir, and tipranavir. Method and composition for the treatment of anemia through the inhibition of furin has been disclosed in WO 2017/147078, which is incorporated herein by reference in its entirety.

In some embodiments, a sample from a subject may be obtained from the subject, and subjected to the assays and methods described herein, for example by measuring an analyte of interest in the sample using the test devices described herein. In some embodiments, the method further comprises methods of preventing, treating, or ameliorating at least one symptom or indication of a coronavirus infection in a subject. In some embodiments, the coronavirus is a SARS-CoV-2 virus. In some embodiments, a subject may have a coronavirus disease, for example, COVID-19. After a positive indication of the coronavirus infection, the method further comprises administering a composition to prevent, treat, or ameliorate at least one symptom or indication of a SARS-CoV-2 infection. In some embodiments, the composition may comprise dexamethasone, remdesivir, an isolated recombinant antibody, an antigen-binding fragment, nelfinavir, or combinations thereof.

### EXAMPLES

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1

### Activating a Substrate

The following example demonstrates a method for activating a substrate.

Regular white printer paper (Up&Up, legal size, 20 weight, brightness of 92) was obtained. A single sheet of paper was placed in a 0.03 M solution of potassium periodate (Acros Organics, Code: 418291000, Lot #A0364464) for two hours at 65° C. The paper was rinsed three times in fresh deionized water for one minute per rinse. The paper was blotted with paper towels and allowed to dry for a minimum of twelve hours at 35° C.

### Example 2

### Preparing a Rollerball Pen with Test Agent

The following example demonstrate a method for preparing antibodies for deposition on activated substrate of Example 1 using a rollerball pen.

Striping the activated substrate of Example 1 with test and control antibodies would traditionally be done with an automated dispenser. However, this example excluded the use of expensive equipment and electricity, since neither are currently feasible in low-resource areas. Even other low-cost options such as using a printer or a simple X-Y plotter required electricity, and it became clear that the target demographic would require a more manual technique. The best way to precisely deliver a substance to a surface without electricity is with a writing instrument. Research led to filling felt-tipped markers, brush pens, ballpoint pens, and paint pens with an antibody solution “ink”, with the most success of delivery being found in a rollerball pen. These rollerball pens can be filled with virtually any antibody ink and are able to draw lines of antibodies onto the activated substrate, producing the control and test lines for many different kinds of tests. This made it easy to test, clean, and reuse the pen with different types of antibodies. This method of delivery promotes user-specific and diverse diagnostic tests for a variety of settings—including low-resource settings and small team operations.

J. Herbin refillable rollerball pens were purchased and the ink was removed. These pens were well suited for delivering antibody ink since they used a wick to draw up the ink and used a refillable piston cartridge to hold ink (Kaweco Mini Piston Converter). The piston cartridge holds the antibody ink, which is carried to the tip of the rollerball pen through a wick when the two are properly connected. Each part can be cleaned and reused with new or different antibody when the user desires. The ink cartridge was rinsed multiple times using 70% EtOH, following by rinsing with water. Once clean, 100 μL of primary test antibodies at a concentration of 1 mg/mL (created from a stock solution of 3.21 mg/mL) was added in a test rollerball pen. A control rollerball pen was similarly prepared, with 100 μL of primary control antibody at 1 mg/mL (created from a stock solution of 2.38 mg/mL) added. The primary test antibodies and the primary control antibodies varies depending on the analyte of interest to be detected. For example, for measuring hCG in a sample, the test antibodies included goat anti-hCG (alpha subunit, made in mouse, MyBiosource, catalog #MBS832263, lot #12401GA09), and the control included goat-anti-mouse antibody (ThermoFisher, catalog #31160, lot #SG2419592).

Additional tests were done to determine the antibody pen's longevity. The antibody pen piston cartridge was filled with 100 μL of rabbit α-goat antibodies, secured to the pen, and then was used to draw several straight lines of antibody ink on a single sheet of paper until the pen ran dry. The paper was then blocked for 1 hour in 5% dry milk solution in TBS-T, washed in deionized water, then incubated with 400 μL AuNP-conjugated goat α-hCG antibodies in TBS under shaking for 1 hour. The paper was removed, rinsed with deionized water, and allowed to dry. The observed lines were measured for length, determining the approximate number of tests 100 μL of antibody in the antibody pen could make. After drawing, blocking, incubating, and drying, the lines were measured to project the total number of tests 100 μL of antibody could produce. A total of 5,588 mm was measured before the integrity of the line began to break up, leaving the rest unmeasured for the sake of quality control. This length would result in approximately 1,117 tests. These tests indicate that the antibody pen is feasible with the low-cost, machine-free manufacturing system required by a low-resource lab or clinic.

### Example 3

### Depositing a Test Agent on the Activated Substrate

The following example demonstrates placement of test and control lines on the activated substrate of Example 1, using the rollerball pens prepared in Example 2.

To the dried activated substrate of Example 1 was applied a test line using the test rollerball pen of Example 2, and a control line using the control rollerball pen of Example 2. The test line was made 5.0 mm apart from the control line, and on the lower third region of the activated substrate. The lower line was the test line, and the upper line was the control line. In traditional LFIs, the absorbance pad causes capillary action. In order to simplify the LFI, the absorbance pad was eliminated, and the test line was positioned near one end of the activated substrate to allow the extra length of paper to facilitate capillary action and improve migration of labeled analyte and detectable label. The test and control lines were applied using a straight edge, a rollerball pen, and approximately 10 μL of a 1 μg/μL antibody solution. More advanced striping systems similar to those used in traditional LFI preparation procedures can be used for commercial tests, but for low resource settings or for in field rapid preparation, the antibody pen method is more cost effective, more accessible, and has greater applicability.

### Example 4

### Blocking the Activated Substrate

The following example demonstrate a method of blocking the activated paper after placement of the test and control lines.

Once the test and control lines were applied, the activated substrate was blocked to prevent non-specific binding. The activated substrate was covered with a blocking solution for one hour using a five percent dry milk solution. After blocking, the substrate was dried at room temperature for two hours. A plastic backing card was attached to the dried substrate and cut into 4.5 mm×33.5 mm test strips for half strips or longer for full strips (4.5 mm×67 mm). Tests strips have been stored at 2-8° C. or room temperature for long term storage.

### Example 5

### Analyzing Analyte of Interest in an Artificial Sample

The following example demonstrates a method of using the test strips of Example 4 for detecting an analyte of interest.

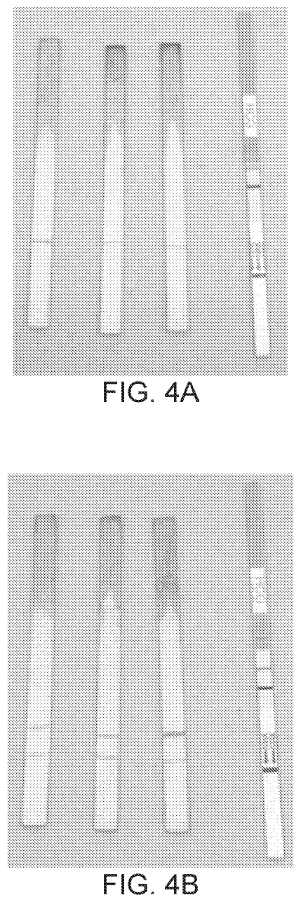

The test strips of Example 4 were striped with goat-anti-mouse antibody (control line) and mouse-anti-hCG antibody (test line) at a 1 mg/mL concentration. The seLFI test was run in a test tube (Falvon, 5 mL, 12×75 mm) with about 100 μL of 5% dry milk in tris-buffered saline with 1% tween, as well as 20 μL of gold nanoparticle-labeled anti-hCG antibody (Catalog #MBS631600, Lot #L17042775). Positive tests were run with a 1 μL spike of hCG at a concentration of 1 mg/mL. Negative tests were run without spiking the buffer with hCG. The results showed successful, specific, and visible binding of the test and control lines, with test results available within 20 minutes. Commercially available pregnancy tests (ClinicalGuard, sensitivity of 25 ng/L) were used as a positive and negative control under comparable conditions. Results are shown in triplicate in FIG. 4A (negative control) and FIG. 4B (positive control), with the commercially available strip shown on the far right in each figure.

Running the tests with positive and negative samples (5% dry milk solution in TBS-T with and without hCG) produced clear, uniform, and legible results (FIGS. 4A and 4B). These results demonstrate the seLFI's ability to compare to traditional LFI results, in speed, reliability, and specificity.

In the positive seLFI results (FIG. 4B), the milk chase buffer, AuNP α-hCG, and hCG were drawn up via capillary action, crossing the entire test, including the test and control lines. The hCG/AuNP α-hCG complex remained bound to the test line, and the AuNP α-hCG remained bound to the control line, resulting in the visual result of two pink lines. In the negative seLFI results (FIG. 4B), the chase buffer and AuNP α-hCG ran through the entire test, crossing both the test and control lines. There was no hCG/AuNP α-hCG complex, so nothing bound to the test line, but the AuNP α-hCG remained bound to the control line, indicating a valid test. This yielded the visual result of one pink line.

### Example 6

### Analyzing Analyte of Interest in a Urine Sample

The following example demonstrates use of the test strips described herein for measuring an analyte of interest in a urine sample.

To run the test strips, 90.0 μL of urine was added to eight 2.0 mL microcentrifuge tubes. For positive samples, 1.0 μL of hCG (1.0 mg/mL) was added to the first four tubes separately and mixed gently with a pipette. An additional positive control was performed with a commercially available pregnancy test, run with the fourth tube according to the package directions. The remaining four tubes were left without hCG as negative controls. An additional negative control was run using a second commercially available pregnancy test. To the six tubes (both positive and negative samples), 10.0 μL of AuNP-conjugated goat α-hCG antibodies were added to each tube separately and mixed gently with a pipette. After preparing these urine samples, previously prepared activated substrates were placed into each tube and allowed to run via capillary action. Production of the tests and the amounts of hCG and AuNP α-hCG remained the same as compared to the tests run in the artificial sample of Example 5. The tests performed exceptionally well, revealing clear test and control lines in the positive samples, and clear control lines in the negative samples. The tests ran significantly faster in urine than they did in milk chase buffer: 2 minutes compared to 20 minutes.

### Example 7

### Analyzing Analyte of Interest in a Serum Sample

The following example demonstrates use of the test strips described herein for measuring an analyte of interest in a serum sample.

To run the test strips, 90.0 μL of fetal bovine serum (FBS) were added to eight 2.0 mL microcentrifuge tubes. For the positive controls, 1.0 μL of hCG (1 mg/mL) was added to the first four tubes separately and mixed gently with a pipette. An additional positive control was performed with a commercially available pregnancy test, run with the sample in the fourth tube according to the package directions. The remaining four tubes were left without hCG as negative controls. An additional negative control was run using a second commercially available pregnancy test. To the six tubes (both positive and negative samples), 10.0 μL of AuNP-conjugated goat α-hCG antibodies were added to each tube separately and mixed gently with a pipette. After preparing these FBS samples, previously prepared activated substrates were placed into each tube and allowed to run via capillary action. Similar to the urine tests, the serum tests functioned properly, presenting consistent positive and negative results, and taking only about 2 minutes to complete. Results for Examples 5, 6, and 7 each appear similar as the results presented in FIGS. 4A and 4B.

### Example 8

### Determining Limits of Detection

The following example demonstrates determination of limits of detection for the activated substrates described herein.

A lateral flow immunoassay must detect a biomarker; furthermore, it must detect the correct levels of that biomarker in the sample. For the detection of analytes of interest, small amounts of biomarker are sometimes present in samples. Thus, determination of a limit of detection is important for determining applicability of the tests described herein. In some instances, the limit of detection is at levels below what can be observed visually by the eye, when using a reader device. However, in many circumstances, the assays described herein will not be used in conjunction with a reader device due to the use of the assays in the field or in low resource environments. A successful outcome for the seLFI would be to detect a concentration of hCG in urine during pregnancy, or about 2.5 μg/mL.

To prepare the test runs, a serial dilution was set up in six dilutions. 90 μL of 5% dry milk solution in TBS-T was added to seven 2 mL microcentrifuge tubes. 10 μL of hCG (1 mg/mL concentration, or 1:1) was added to and mixed in the first dilution, resulting in a to 10 dilution of hCG concentration. 10 μL of solution was removed from the 1 to 10 dilution, added to the second microcentrifuge tube and gently mixed, resulting in a 1 to 100 hCG concentration. This process was repeated until six tubes contained hCG ranging from a 10 fold dilution to a 1 million dilution (0.1 mg/mL; 0.01 mg/mL; 1 μg/mL; 0.1 μg/mL: 0.01 μg/mL; 1 ng/mL). The final tube was left without hCG and served as a negative control.

This serial dilution was repeated three times in additional microcentrifuge tubes: two of the sets of serial dilutions acted as replicates for the original dilution, and one set of serial dilutions was used to run commercially available pregnancy tests as a positive control. These commercially available tests were run according to the package directions, using each tube separately for a total of six positive control tests. The final tube, having no hCG, served as the negative control.

In the remaining three sets of tubes, 10 μL of AuNP-conjugated goat α-hCG was added to and gently mixed in each separate dilution. After preparing these dilution samples, previously prepared activated substrates were placed into each tube and allowed to run via capillary action.

This experimental set-up was repeated to test the seLFI's limit of detection in urine, replacing the milk chase buffer volume with the same volume of urine, keeping all other conditions the same.

Finding the seLFI's limit of detection allow for a side-by-side comparison of the seLFI's sensitivity and the sensitivity of commercially available tests. Ideally, the seLFI would be able to detect hCG at the level of 1 to 10,000 in order to detect 2.5 ng/mL of hCG in a positive urine sample. The seLFI consistently bound hCG at concentrations of 1 to 100 (0.01 mg/mL), to 1 to 1000 (1 μg/mL), with unreliable, faint binding at 1 to 10,000 (0.1 μg/mL).

Sensitivity of the assays described herein are improved by increasing the amount of AuNP α-hCG used in the assay. Increased AuNP saturate the available hCG, increasing the intensity of the test line, despite the lower levels of protein in the sample. The sensitivity is also increased by optimizing the antibody-antigen pairings. With a fade-out at 1 to 10,000 concentration of hCG, the issue is between the α-hCG, hCG, and AuNP α-hCG sandwich complex. Sensitivity can also be increased by increasing the concentration of α-hCG on the test line, so as to capture more hCG-AuNP α-hCG complexes, raising the level of sensitivity. Finally, sensitivity can also be increased by adding silver staining of the gold nanoparticles, which cause an enhanced plasmon resonance, capable of increasing the limit of detection 10 to 100 fold.

### Example 9

### Analyzing Viral Particles in a Sample

The following example demonstrates use of the test strips provided herein for measuring viral particles in a sample.

The activated substrates disclosed herein can be used for early diagnostic use for emerging outbreaks. This is possible because a functioning activate substrate can be rapidly built on site starting with printer paper, a plastic backing card, and an antibody pen, making the activated substrate a rapidly deployable emergency use test for any application as long as antibodies are available.

A sample from a subject having or suspected of having a viral infection is obtained. The viral infection could be an infection from a rhinovirus, seasonal influenza, respiratory syncytial virus, hepatitis A, norovirus, rotavirus, influenza virus, SARS virus, MERS, influenza a, influenza b, cold virus. The sample is contacted with the detectable label, and allowed to incubate for a period of time sufficient to label viral particles associated with the viral infection. The test strips disclosed herein are contacted with the labeled sample, and sample is allowed to flow through the test strip to the test and control lines. If a viral infection is present, a visually detectable signal will be detected at the test line.

In these examples, such as for testing SARS-CoV-2, a protein line is deposited on the test strip, and the serological test include bound conjugated antibodies in the sample that bind to the test protein line. Covid-19 has a remarkably diverse effect on infected individuals with symptoms ranging from no symptoms to respiratory failure and death. Testing is critical to protect the high-risk population. Nasal swabs are painful and inconvenient and this sample collection exposes medical personnel to potentially infected individuals. An at home, point of care diagnostic saliva test minimizes exposure of medical staff. Individuals that have been exposed to someone with Covid-19 can quarantine and perform self-testing during the quarantine period to observe if/when they become contagious. At home, POC tests can minimize exposure of high-risk individuals if everyone had access to simple seLFI tests. The seLFI tests will help identify Covid-19 positive individuals so they can avoid contact with high-risk individuals; and 2) early diagnosis of Covid-19 positive high-risk patients so they can receive treatment to minimize the progression of Covid-19 to minimize hospitalization and ICU admittance.

Upon a positive test assay, the preventative Covid-19 treatments are initiated, administered, or recommended, such as Viracept, Dexamethasone, or Remdesivir as a treatment to prevent progression of Covid-19.

The test strips described herein can further be used for detecting any biomarker of interest to determination of the presence of the biomarker in a sample. The test strips may be quickly and efficiently prepared to meet any need on a fast time scale, such as for outbreaks, or for regions that lack specialized equipment. For example, the test strips may be used to distinguish between parasitic anemia and nutritional anemia; for the development of test assays for developing countries and for individualized diagnosis; to distinguish between dietary anemia and anemia caused by chronic inflammation; for infectious diseases; for evaluating nutritional status; for detecting causes of allergies; or for aiding in the general health safety diagnostics.

The previous description of the disclosed implementations is provided to enable any person skilled in the art to make or use the present invention. Various modifications to these implementations will be readily apparent to those skilled in the art, and the generic principles defined herein may be applied to other implementations without departing from the spirit or scope of the invention. Thus, the present invention is not intended to be limited to the implementations shown herein but is to be accorded the widest scope consistent with the principles and novel features disclosed herein.

What is claimed is:

1. A test strip comprising:
a flow path configured to receive a fluid sample, wherein the flow path comprises activated substrate that has been blocked with a blocking agent; and
a test line coupled to the flow path, wherein the test line comprises immobilized test agent specific to an analyte of interest.

2. The test strip of claim 1, wherein the activated substrate is aldehyde functionalized paper.

3. The test strip of any one of claims 1-2, wherein the activated substrate is activated with potassium periodate.

4. The test strip of any one of claims 1-3, wherein the blocking agent is bovine serum albumin, casein, or a solution of powdered milk.

5. The test strip of any one of claims 1-4, wherein the test agent is an antibody or a protein specific to the analyte of interest.

6. The test strip of any one of claims 1-5, wherein the flow path is configured to receive a fluid sample comprising labeled analyte of interest, wherein the labeled analyte of interest is labeled with a detectable label.

7. The test strip of any one of claims 1-6, further comprising a control line coupled to the flow path, wherein the control line comprises immobilized control agent specific to a detectable label.

8. The test strip of any one of claims 6-7, wherein the detectable label comprises a metal nanoparticle conjugated to an antibody specific to the analyte of interest.

9. The test strip of claim 8, wherein the metal nanoparticle is a gold nanoparticle.

10. The test strip of any one of claim 1-9, further comprising a backing card.

11. The test strip of any one of claims 1-10, wherein the fluid sample is selected from the group consisting of a blood, plasma, urine, sweat, nasal, lacrimal, or saliva sample.

12. A kit comprising:
a substrate;
an activation reagent;
a dispensing device comprising test agent specific to an analyte of interest;
a blocking reagent; and
a detectable label specific to the analyte of interest.

13. The kit of claim 12:
wherein the substrate is printer paper;
wherein the activation reagent is potassium periodate;
wherein the dispensing device is a rollerball pen comprising the test agent;
wherein the test agent is an antibody or protein specific to the analyte of interest;
wherein the blocking reagent is bovine serum albumin, casein, or a solution of powdered milk; and
wherein the detectable label is a gold nanoparticle conjugated to an antibody specific to the analyst of interest.

14. The kit of any one of claims 12-13, further comprising a control dispensing device comprising a control agent.

15. The kit of any one of claims 12-14, further comprising a backing card.

16. A method of manufacturing a test strip comprising:
a) contacting a substrate with an activation reagent to generate activated substrate;
b) contacting the activated substrate with a test agent at a test line; and
c) contacting the activated substrate after b) with a blocking reagent.

17. The method of claim 16, wherein contacting the activated substrate with the test agent comprises applying the test agent to the activated substrate using a dispensing device.

18. The method of claim 17, wherein the dispensing device is a pen or marker comprising the test agent.

19. The method of any one of claims 17-18, wherein the dispensing device is a rollerball pen.

20. The method of any one of claims 16-19, wherein the activation reagent is potassium periodate.

21. The method of any one of claims 16-20, wherein the test agent comprises an antibody or protein specific to the analyte of interest.

22. The method of any one of claims 16-21, wherein the blocking reagent comprises bovine serum albumin, casein, or a solution of powdered milk.

23. The method of any one of claims 16-22, further comprising contacting the activated substrate after b) with a control agent at a control line.

24. The method of claim 23, wherein the control agent comprises an antibody or protein specific to a detectable label.

25. The method of claim 24, wherein the detectable label comprises a metal nanoparticle conjugated to an antibody specific to the analyte of interest.

26. The method of claim 25, wherein the metal nanoparticle is a gold nanoparticle.

27. The method of any one of claims 16-26, wherein the substrate is paper.

28. The method of claim 16, comprising:
soaking paper in 0.03 M potassium periodate to generate activated paper;
applying an antibody or protein specific to analyte of interest having a concentration of about 1 mg/mL to the activated paper using a first rollerball pen;
applying an antibody or protein specific to a detectable label having a concentration of about 1 mg/mL to the activated paper using a second rollerball pen; and
soaking the activated paper in a solution of 5% powdered milk.

29. The method of any one of claims 16-28, further comprising applying a backing card to the activated substrate, and cutting the activated substrate into test strips.

30. A method for measuring an analyte in a fluid sample, comprising:
providing a fluid sample having or suspected of having an analyte of interest;
contacting the fluid sample with a detectable label that specifically binds analyte of interest, wherein the detectable label binds analyte of interest in the fluid sample to form a labeled analyte of interest;
contacting a test strip with a sample, wherein the test strip comprises:
a flow path configured to receive a fluid sample, wherein the flow path comprises activated substrate that has been blocked with a blocking agent; and
a test line coupled to the flow path, and comprising immobilized test agent specific to an analyte of interest;
flowing the sample through the test strip;
binding the labeled analyte of interest to the immobilized test agent at the test line; and
detecting a signal from the labeled analyte of interest bound to the immobilized test agent at the test line.

31. The method of claim 30, wherein the detected signal is an optical signal.

32. The method of any one of claims 30-31, wherein the analyte of interest is a protein or a viral particle.

33. The method of any one of claims 30-32, wherein the detectable label comprises a metal nanoparticle conjugated to an antibody that specifically binds analyte of interest.

34. The method of claim 33, wherein the metal nanoparticle is a gold nanoparticle.

35. The method of any one of claims 30-34, wherein the sample is selected from the group consisting of a blood, plasma, urine, sweat, nasal, lacrimal, or saliva sample.

36. The method of any one of claims 30-35, further comprising comparing an intensity of the signal at the test line to an intensity of a control signal of known concentrations of analyte of interest.

37. The method of any one of claims 30-36, further comprising increasing an intensity of the signal at the test line by incubating the test strip in a signal enhancing solution.

38. The method of claim 37, wherein the signal enhancing solution is a solution of silver.
